(19) United States
(12) Patent Application Publication
(10) Pub. No.: US 2023/0260424 A1
(43) Pub. Date: Aug. 17, 2023

(54) METHOD AND APPARATUS FOR MANAGING NUTRITION AND CHRONIC DISEASE, AND STORAGE MEDIUM AND TERMINAL DEVICE

(71) Applicant: HO YAN TSUI, HONG KONG (CN)

(72) Inventor: HO YAN TSUI, HONG KONG (CN)

(21) Appl. No.: 18/014,409

(22) PCT Filed: Aug. 23, 2021
(86) PCT No.: PCT/CN2021/114017

§ 371 Date: Jan. 4, 2023

(30) Foreign Application Priority Data
Jul. 8, 2020 (HK) 32020010819.1
Mar. 29, 2021 (CN) 202110333270.4

(51) Int. Cl. G09B 19/00; G16H 20/60

(52) U.S. Cl. CPC G09B 19/0092; G16H 20/60

(57) ABSTRACT

A method and an apparatus for managing nutrition and a chronic disease, and a storage medium and a terminal device. The method comprises: acquiring personal detail information of a user, and on the basis of the personal detail information, calculating nutritional requirement information for the user (S101), the personal detail information comprising chronic disease information of the user, and the nutrition requirement information comprising a user calorie requirement and a required amount of nutrients that impact the user's chronic disease; acquiring the names of one or more foods the user wishes to consume (S102); on the basis of the names of the one or more foods the user wishes to consume, acquiring calorie and nutrient content information of each food among the one or more foods, and on the basis of the nutrition requirement information and the calorie and nutrient content information of each food, calculating a suggested consumption portion for each food (S103); and outputting the suggested consumption portions (S104). In the present technical solution for managing nutrition and chronic diseases, comprehensive nutritional guidance and chronic disease management can be performed for a user with a chronic disease, establishing and improving a healthy lifestyle for the user with a chronic disease. (FIG. 1)

The present application claims the priority of a Hong Kong patent application with the application number of 32020010819.1 and the title of “METHOD AND APPARATUS FOR MANAGING NUTRITION AND CHRONIC DISEASE, AND STORAGE MEDIUM AND TERMINAL DEVICE” filed with the Intellectual Property Office of the Hong Kong Special Administrative Region on Jul. 8, 2020, and the priority of a Chinese patent application with the application number of 202110333270.4 and the title of “METHOD AND APPARATUS FOR MANAGING NUTRITION AND CHRONIC DISEASE, AND STORAGE MEDIUM AND TERMINAL DEVICE” filed with the China National Intellectual Property Administration (“CNIPA”) on Mar. 29, 2021. The full contents thereof are incorporated in the present application by reference.

### FIELD OF THE INVENTION

The present application relates to the technical field of intelligent devices, and in particular to a nutrition and chronic disease management method, apparatus, storage medium and terminal equipment.

### BACKGROUND OF THE INVENTION

Modern society has a rapid pace and high pressure of life. There is a high prevalence of chronic diseases, especially among the elderly. Studies have shown that the chronic diseases are closely associated with bad lifestyle. Among them, the irregular diet, unreasonable diet arrangement, diet misconceptions, high fat, high heat and accumulation of toxin caused by long-term intake of garbage food are all the causes of chronic diseases. The regular diet, balanced diet, targeted intake of calories and nutrients required by the chronic diseases will help to avoid illness from the mouth, effectively alleviate or even inhibit the development of human chronic diseases.

There are a number of nutritional guidance software in the prior art that provide dietary guidance to the general public. However, the existing nutritional software usually has the following defects: The service object of nutritional software is the public instead of individuals with chronic diseases. The nutritional guidance generally provides the guidance on calories, but the types of nutrients involved are rarely involved, and some only involve one or two nutrient ingredients. The dietary recommendation provided is only an estimation result rather than an accurate calculation result, and the user has a long waiting time when the dietary recommendation is provided. Most nutritional software only provides pre-meal recommendation, or only provides post-meal supervision. There is no nutritional guidance software that can provide both the pre-meal recommendation and the post-meal supervision. Furthermore, the nutritional guidance software of the prior art is poor in user stickiness because the dietary guidance cannot be made both long-term and targeted for different users.

In view of the above, it would be an urgent concern for a person skilled in the art to provide a method, apparatus, terminal equipment, etc. for providing comprehensive nutrition guidance and chronic disease management for a user suffering from chronic diseases.

### SUMMARY OF THE INVENTION

It is an object of the present application to provide a nutrition and chronic disease management method, apparatus, storage medium and terminal equipment, so as to provide a comprehensive nutrition guidance and chronic disease management for, and establish and improve a healthy life mode of a user suffering from the chronic diseases.

In order to solve the above technical problem, in a first aspect, the present application discloses a nutrition and chronic disease management method, comprising the following steps S101 to S104:

S101: acquiring personal profile information of a user, and calculating nutritional requirement information of the user according to the personal profile information, wherein the personal profile information includes chronic disease information of the user, and the nutritional requirement information includes the calories required by the user and the amounts of nutrients affecting the chronic diseases of the user;

S102: obtaining a name of one or more foods to be taken by the user;

S103: obtaining caloric and nutrient content information of each of the one or more foods according to the name of the one or more foods, and calculating a recommended serving size of each food according to the nutritional requirement information and the caloric and nutrient content information of each food; and

S104: outputting the recommended serving size.

Optionally, the personal profile information further includes personal physical information, the personal physical information including age, gender, height, weight, personal exercise amount, and eating habit information of the user; and the calories required by the user are calculated based on the personal physical information.

Optionally, the nutritional requirement information is for a current meal period determined according to the user's daily nutritional requirement information and the user's eating habits.

Optionally, the chronic disease information includes chronic disease medical record information and chronic disease history information, wherein the chronic disease medical record information includes the medical record data of one to three chronic diseases of the user. In order to ensure the accuracy and pertinence of calculation, it is recommended that the types of chronic diseases input by the user should be three or less. The chronic disease history information includes the disease type, diagnosis time, severity, treatment condition, and used drugs of the chronic disease of the user.

Optionally, the method further comprises: obtaining a predetermined serving size of each of the one or more foods when obtaining the name of the one or more foods to be taken by the user, and calculating a recommended serving size of each food according to the nutritional requirement information and the caloric and nutrient content information of each food, comprising: adjusting the predetermined serving size according to the nutritional requirement information and the caloric and nutrient content information of each food to obtain the recommended serving size.

Optionally, the nutrients affecting the chronic diseases of the user include at least eighteen of the following: protein, carbohydrate, fat, dietary fiber, sugar, saturated fat, trans fat, cholesterol, calcium, copper, iron, magnesium, manganese, phosphorus, potassium, sodium, zinc and vitamin C. If there is a need for nutrients other than these eighteen types, they can be added as needed.

Optionally, the method further comprises: selecting three nutrients most affecting the chronic diseases of the user from the nutrients affecting the chronic diseases of the user, and using the required amounts of the three nutrients and the calories required by the user as four top priorities for guiding the user's meal. For example, for a patient suffering from a certain chronic disease, the protein, dietary fiber and cholesterol may be selected from the above at least eighteen nutrients as the three nutrients most affecting the chronic disease, together with the calories required by the user as the four top priorities for guiding the user's meal.

Optionally, the method further comprises: obtaining a predetermined serving size of each of the one or more foods when obtaining the name of the one or more foods to be taken by the user, and calculating a recommended serving size of each food according to the nutritional requirement information and the caloric and nutrient content information of each food, comprising: adjusting the predetermined serving size of each food respectively according to the four top priorities to obtain the recommended serving size of each food.

Optionally, the adjustment of the predetermined serving size of each food according to the calories required by the user is carried out at the end of the four top priorities, so as to make the total calories contained in the recommended serving sizes equal to the calories required by the user.

Optionally, when the predetermined serving size of each food is obtained, the predetermined upper and lower threshold values of adjustment for the predetermined serving size of each food are also obtained, so as to avoid that a particular type of dish or ingredient reaches a very unreasonable serving size by addition and reduction when adjusting the serving size predetermined by the user.

Optionally, the caloric and nutrient content information of each of the one or more foods obtained according to the name of the one or more foods includes: the caloric and nutrient content information of each food obtained from a predetermined recipe nutrient database according to the name of the one or more foods.

Optionally, the method further comprises after the meal, recording the serving sizes taken by the user greater or smaller than the recommended serving sizes, so as to guide the user's subsequent meals; generating a dietary record report of the user according to a predetermined first period (e.g., one month as a period), and determining whether the user has an insufficient or excessive nutrient intake during the predetermined first period, so as to determine whether to give a dietary remedy recommendation; or providing a dietary recommendation to the user according to a predetermined second period (e.g., a quarter or year as a period).

In a second aspect, the present application also discloses a nutrition and chronic disease management apparatus 200, comprising: a personal information acquisition module 201, a food-to-take acquisition module 202, a dietary recommendation calculation module 203, and an output module 204.

Amongst them, the personal information acquisition module 201 can be used for acquiring the personal profile information of the user, and calculating the nutritional requirement information of the user according to the personal profile information, wherein the personal profile information includes the chronic disease information of the user, and the nutritional requirement information includes the calories required by the user and the amounts of nutrients affecting the chronic diseases of the user.

The food-to-take acquisition module 202 can be used for acquiring the name of one or more foods to be taken by the user.

The dietary recommendation calculation module 202 can be used for obtaining caloric and nutrient content information of each of the one or more foods according to the name of the one or more foods, and calculating a recommended serving size of each food according to the nutritional requirement information and the caloric and nutrient content information of each food.

The output module 204 can be used for outputting the recommended serving size.

Optionally, the personal profile information further includes the personal physical information, the personal physical information including age, gender, height, weight, personal exercise amount, and eating habit information of the user; and the calories required by the user which are calculated based on the personal physical information.

Optionally, the nutritional requirement information is for the current meal period determined according to the user's daily nutritional requirement information and the user's eating habits.

Optionally, the chronic disease information includes chronic disease medical record information and chronic disease history information, wherein the chronic disease medical record information includes the medical record data of one to three chronic diseases of the user, and the chronic disease history information includes the disease type, diagnosis time, severity, treatment condition, and used drugs of the chronic disease of the user.

Optionally, the food-to-take acquisition module 202 is also applied to: obtaining a predetermined serving size of each of the one or more foods when obtaining the name of the one or more foods to be taken by the user, and the dietary recommendation calculation module 202 is also applied to: adjusting the predetermined serving size according to the nutritional requirement information and the caloric and nutrient content information of each food, so as to obtain the recommended serving size.

Optionally, the nutrients affecting the chronic diseases of the user include at least eighteen of the following: protein, carbohydrate, fat, dietary fiber, sugar, saturated fat, trans fat, cholesterol, calcium, copper, iron, magnesium, manganese, phosphorus, potassium, sodium, zinc and vitamin C.

Optionally, the personal information acquisition module 201 is also applied to: selecting three nutrients most affecting the chronic diseases of the user from the nutrients affecting the chronic diseases of the user, and using the required amounts of the three nutrients and the calories required by the user as four top priorities for guiding the user's meal.

Optionally, the food-to-take acquisition module 202 is also applied to: obtaining a predetermined serving size of each of the one or more foods when obtaining the name of the one or more foods to be taken by the user, and the dietary recommendation calculation module 202 is also applied to: adjusting the predetermined serving size of each food respectively according to the four top priorities to obtain the recommended serving size of each food.

Optionally, the dietary recommendation calculation module 202 is also applied to adjusting the predetermined serving size of each food according to the calories required by the user at the end of the four top priorities, so as to make the total calories contained in the recommended serving sizes equal to the calories required by the user.

Optionally, the food-to-take acquisition module 202 is also applied to obtaining the predetermined upper and lower threshold values of adjustment for the predetermined serving size of each food, when the predetermined serving size of each food is obtained. The purpose of setting the predetermined upper and lower threshold values of adjustment is to avoid adding or reducing certain ingredient to an unreasonable amount in the process of counter-claim calculation based on the top priorities.

Optionally, the dietary recommendation calculation module 202 is also applied to obtaining the caloric and nutrient content information of each food from a predetermined recipe nutrient database according to the name of the one or more foods.

Optionally, the nutrition and chronic disease management apparatus further comprises a supervision and recommendation module for recording the serving sizes taken by the user greater or smaller than the recommended serving sizes after the meal, so as to guide the user's subsequent meals.

Optionally, the supervision and recommendation module is also applied to generating a dietary record report of the user according to a predetermined first period (e.g., one month as a period), and determining whether the user has an insufficient or excessive nutrient intake during the predetermined first period, so as to determine whether to give a dietary remedy recommendation. In addition, the supervision and recommendation module is also applied to providing a dietary recommendation to the user according to a predetermined second period (e.g., a quarter or year as a period).

In a third aspect, the present application also discloses a storage medium storing a program. When the program is executed, the instructions for implementing any one of the above nutrition and chronic disease management methods can be executed.

In a fourth aspect, the present application also discloses a terminal equipment 300 comprising a readable medium 301 storing the program instructions and a processor 302. When the processor 302 executes any one of the above nutrition and chronic disease management methods when running the program instructions.

According to the technical solution of the nutrition and chronic disease management method, apparatus, storage medium and terminal equipment of the present application, the dietary guidance of the present application can be specially designed for the users with chronic diseases, and can establish and improve the healthy life mode of the users with chronic diseases. The large database resources can be guaranteed by the database of independently developed recipes, including the combination of Chinese and western cuisines, and the public food safety database of the country or region (such as the database of Hong Kong Food Safety Center in Hong Kong). In addition, when the technical solutions of the embodiments of the present application provide a dietary recommendation, the caloric requirements and various nutrient requirements of a user are considered comprehensively, e.g., at least eighteen types of nutrients may be included. Also, more accurate and scientific dietary guidance can be provided by focusing on the three nutrients that have the greatest impact on the user's chronic diseases. The dietary recommendation provided in the technical solution of the present application is an accurate calculation result rather than a simple estimation. The recommendation can be quickly finished according to the menu provided by the user, and the user does not need to wait for a long time. The technical solution of the present application can not only provide the pre-meal recommendation, but also perform the post-meal supervision. It can supervise the diet of a user for long term, and pertinently provide the personalized education for different users.

### BRIEF DESCRIPTION OF THE DRAWINGS

In order to illustrate the technical solutions in the prior art and the embodiments of the present application more clearly, a brief introduction is now provided for the drawings to be used in the prior art and the embodiments of the present application. Certainly, the following illustration of the drawings relating to the embodiments of the present application is merely a few of the embodiments of the present application. A person of ordinary skill in the art would be able to obtain other drawings according to the provided drawings without any inventive effort. The obtained other drawings also fall within the protection scope of the present application.

FIG. 1 is a flow chart of a nutrition and chronic disease management method disclosed in an embodiment of the present application;

FIG. 2 is a schematic view of the structure of a nutrition and chronic disease management apparatus disclosed in an embodiment of the present application; and

FIG. 3 is a schematic view of the structure of a terminal equipment disclosed in an embodiment of the present application.

### DETAILED DESCRIPTION OF THE PREFERRED EMBODIMENT

A essence of the present application is to provide a nutrition and chronic disease management method, apparatus, storage medium and terminal equipment, so as to provide the users suffering from chronic diseases with a comprehensive nutrition guidance and chronic disease management and help them to establish and improve a healthy life mode.

In order to more clearly and completely elaborate the technical solutions in the embodiments of the present application, they will be elaborated below with reference to the figures in the embodiments of the present application. Apparently, the embodiments disclosed are only a few, but not all embodiments of the present application. Based on the embodiments in the present application, all the other embodiments obtained by a person of ordinary skill in the art without making any inventive effort fall within the scope of protection of the present application.

As disclosed in the background of the present application, the nutritional guidance software of the prior art has various drawbacks, and cannot provide comprehensive nutritional advice and chronic disease management for users with chronic diseases. The embodiments of the present application provide a technical solution of comprehensive nutrition and chronic disease management for users suffering from chronic diseases. By using a developed software, Nutrition plus Chronic Disease Management Application (“NCMA”), a pre-meal dietary recommendation is provided before a meal according to the menu that the user intends to have, and a supervision is performed after the meal according to the meal feedback input by the user to supervise whether the user has an excessive or insufficient intake of calories and nutrients, so as to combine a pre-meal notice and a post-meal supervision. Amongst them, the NCMA can be installed on various terminal equipment for use, including, but not limited to, various terminal equipment such as a mobile phone, a laptop, a tablet computer, a desktop computer, and a vehicle-mounted computer. For the convenience of elaboration, the embodiments of the present application mainly take the use of NCMA installed in a mobile terminal such as a mobile phone as an example, but it is not intended to limit the technical solution of the present application to be applied only in a mobile phone terminal.

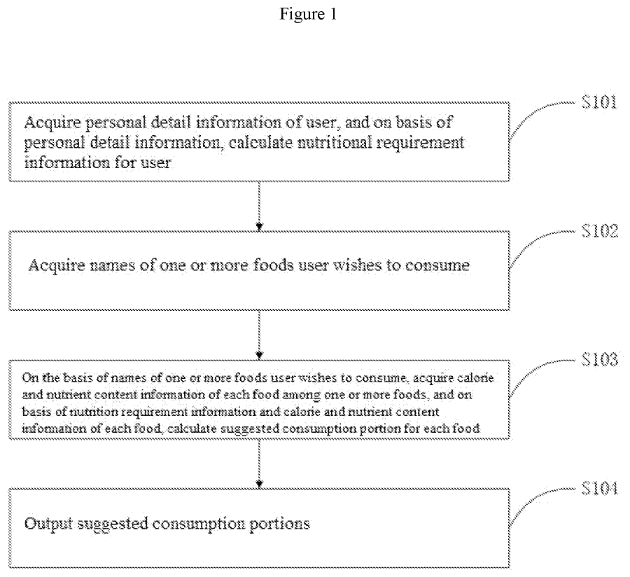

FIG. 1 is a flow chart of a nutrition and chronic disease management method disclosed in an embodiment of the present application. Referring to FIG. 1, the nutrition and chronic disease management method according to an embodiment of the present application comprises the following steps S101 to S104:

S101: acquiring personal profile information of a user, and calculating nutritional requirement information of the user according to the personal profile information, wherein the personal profile information includes chronic disease information of the user, and the nutritional requirement information includes the calories required by the user and the amounts of nutrients affecting the chronic diseases of the user;

S102: obtaining a name of one or more foods to be taken by the user;

S103: obtaining caloric and nutrient content information of each of the one or more foods according to the name of the one or more foods, and calculating a recommended serving size of each food according to the nutritional requirement information and the caloric and nutrient content information of each food; and

S104: outputting the recommended serving size.

Amongst them, the personal profile information further includes personal physical information, the personal physical information including age, gender, height, weight, personal exercise amount, and eating habit information of the user; and the calories required by the user are calculated based on the personal physical information.

The nutritional requirement information is for a current meal period determined according to the user's daily nutritional requirement information and the user's eating habits.

The chronic disease information includes chronic disease medical record information and chronic disease history information, wherein the chronic disease medical record information includes the medical record data of one to three chronic diseases of the user. In order to ensure the accuracy and pertinence of calculation, it is recommended that the types of chronic diseases input by the user should be three or less. The chronic disease history information includes the disease type, diagnosis time, severity, treatment condition, and used drugs of the chronic disease of the user.

In addition, the nutrition and chronic disease management method according to an embodiment of the present application may further comprise: obtaining a predetermined serving size of each of the one or more foods when obtaining the name of the one or more foods to be taken by the user, and calculating a recommended serving size of each food according to the nutritional requirement information and the caloric and nutrient content information of each food, comprising: adjusting the predetermined serving size according to the nutritional requirement information and the caloric and nutrient content information of each food to obtain the recommended serving size.

In an embodiment of the present application, the nutrients affecting the chronic diseases of the user include at least eighteen of the following: protein, carbohydrate, fat, dietary fiber, sugar, saturated fat, trans fat, cholesterol, calcium, copper, iron, magnesium, manganese, phosphorus, potassium, sodium, zinc and vitamin C. If the nutrients other than these eighteen types are required according to actual needs, they can be added according to the calculated requirements.

In one embodiment of the present application, the nutrition and chronic disease management method further comprises: selecting three nutrients most affecting the chronic diseases of the user from the nutrients affecting the chronic diseases of the user, and using the required amounts of the three nutrients and the calories required by the user as four top priorities for guiding the user's meal. For example, for a patient suffering from a certain chronic disease, the protein, dietary fiber and cholesterol may be selected from the above at least eighteen nutrients as the three nutrients most affecting the chronic disease, together with the calories required by the user as the four top priorities for guiding the user's meal.

Accordingly, the nutrition and chronic disease management method of an embodiment of the present application further comprises: obtaining a predetermined serving size of each of the one or more foods when obtaining the name of the one or more foods to be taken by the user, and calculating a recommended serving size of each food according to the nutritional requirement information and the caloric and nutrient content information of each food, comprising: adjusting the predetermined serving size of each food respectively according to the four top priorities to obtain the recommended serving size of each food.

Amongst them, the adjustment of the predetermined serving size of each food according to the calories required by the user is carried out at the end of the four top priorities, so as to make the total calories contained in the recommended serving sizes equal to the calories required by the user.

In the nutrition and chronic disease management method of an embodiment of the present application, when the predetermined serving size of each food is obtained, the predetermined upper and lower threshold values of adjustment for the predetermined serving size of each food are also obtained, so as to avoid that a particular type of dish or ingredient reaches a very unreasonable serving size by addition and reduction when adjusting the serving size predetermined by the user.

In an embodiment of the present application, the caloric and nutrient content information of each of the one or more foods can be obtained according to the name of the one or more foods from the predetermined recipe nutrient database. The predetermined recipe nutrient database can be a database of independently developed recipes, including the combination of Chinese and western cuisines, and the public food safety database of the country or region (such as the database of Hong Kong Food Safety Center in Hong Kong), so as to provide large database resources.

The nutrition and chronic disease management method of an embodiment of the present application further comprises after the meal, recording the serving sizes taken by the user greater or smaller than the recommended serving sizes, so as to guide the user's subsequent meals; generating a dietary record report of the user according to a predetermined first period (e.g., one month as a period), and determining whether the user has an insufficient or excessive nutrient intake during the predetermined first period, so as to determine whether to give a dietary remedy recommendation; or providing a long term dietary recommendation to the user according to a predetermined second period (e.g., a quarter or year as a period).

According to the technical solution of a nutrition and chronic disease management apparatus in an embodiment of the present application, the dietary guidance in the embodiment of the present application can be specially designed for the user with chronic diseases, and can establish and improve the healthy life mode of the user with chronic diseases. The large database resources can be guaranteed by the database of independently developed recipes, including the combination of Chinese and western cuisines, and the public food safety database of the country or region (such as the database of Hong Kong Food Safety Center in Hong Kong). In addition, when the technical solution of the embodiment of the present application provides a dietary recommendation, the caloric requirements and various nutrient requirements of the user are considered comprehensively, e.g., at least eighteen types of nutrients may be included. Also, more accurate and scientific dietary guidance can be provided by focusing on the three nutrients that have the greatest impact on the user's chronic diseases. The dietary recommendation provided in the technical solution of the present application is an accurate calculation result rather than a simple estimation. The recommendation can be quickly finished according to the menu provided by the user, and the user does not need to wait for a long time. The technical solution of the present application can not only provide the pre-meal recommendation, but also perform the post-meal supervision. It can supervise the diet of a user for long term, and pertinently provide the personalized education for different users.

The use of NCMA and dietary guidance in the embodiments of the present application consider the following points comprehensively to manage the nutrition and chronic diseases of the users:

Knowing the user: The nutritional requirement information includes the personal physical requirements of each user, the dietary requirements for the chronic diseases, and the at least eighteen nutrients and calories required by the user each day;

Knowing the nutrients: The medical nutrition and database of independently developed various Chinese and western cuisines are based on;

Accurate algorithm: The appropriate diet quantities per meal for each user are calculated by using the program instructions and a predetermined algorithm covering calories and at least eighteen nutrients (“eighteen algorithm”) to closely managing the health of each user and control the chronic diseases;

Among them, the eighteen algorithm refers to the calculation using the calories required by the user and at least eighteen nutrients, because controlling the intake of calories can control the user's weight, and taking appropriate amounts of nutrients such as protein can help the body to stay healthy. The embodiments of the present application adopt the calories and eighteen nutrients beneficial to the human body as the calculation factors to ensure that the dietary recommendation finally provided is scientific and reasonable.

Pre-meal notice: In the application process, the user reports a menu intended to eat before a meal. The NCMA quickly calculates an appropriate amount of each food in the menu according to personal requirements, i.e., forming a pre-meal notice, and providing the pre-meal report to the user to guide the user's meal;

Post-meal review: After a meal, the user may use the NCMA for post-meal review. The NCMA feeds back the type and amount of food taken insufficiently or excessively, and suggests the compensation for eating more or less during subsequent meals. The NCMA may generate a monthly dietary record report based on the user's monthly dietary record. It can be seen from the monthly report whether the user's intake of certain nutrients this month is insufficient, and needs to be remedied, and how to remedy it;

Education and help: In addition, the NCMA may provide long-term educational material to help the users understand the dietary quality, establish and improve a healthy lifestyle.

As can be seen from the above, the use of NCMA in the embodiments of the present application can provide great help to the public, especially the elderly and patients with chronic diseases.

The technical solutions of the nutrition and chronic disease management method in the embodiments of the present application are illustrated in detail below with reference to specific examples.

(1) Knowing the user: acquiring personal profile information of a user, and calculating nutritional requirement information of the user according to the personal profile information, wherein the personal profile information includes chronic disease information of the user, etc.

In the embodiments of the present application, the NCMA may be used to obtain the calories and serving sizes of eighteen nutrients for each user every day.

After the user downloads the NCMA, the following personal physical information data is required to be entered to calculate the nutritional requirement information data. The personal physical information data includes, but is not limited to, 1) age, gender, height, weight, personal exercise amount, eating habits, etc.; 2) the chronic disease information includes chronic disease medical record information and chronic disease history information.

Among them, acquiring the personal exercise amount of the user can be performed by, but is not limited to, a questionnaire survey on the user. The personal exercise amount may include the following selectable levels: sedentariness (little exercise), mild exercise (exercise 1-3 days per week), moderate exercise (moderate exercise and/or physical exercise for 3-5 days per week), severe exercise (vigorous exercise for 6-7 days per week), high intensity exercise (physical work or physical training such as twice training per day). The questions are not limited to the above types. A person skilled in the art can flexibly set the contents of a questionnaire according to the requirements, as long as the personal exercise amount of the user can be comprehensively obtained for guiding a reasonable diet in each meal of the user.

Acquiring the eating habits of a user can be performed by, but is not limited to, a questionnaire survey on the user: 1. Among the three meals a day, which meal is intended as the main meal. Options include breakfast, lunch, and dinner. 2. Do you have a habit of eating snacks? Options may include no, rarely, and yes (afternoon snack or night snack). 3. If your body weight is too high to control chronic disease, do you want to reduce the nutritional intake to control the body weight? Options include yes and no. The questions are not limited to the above three. A person skilled in the art can flexibly set the contents of a questionnaire according to the requirements, as long as the eating habits of the user can be comprehensively obtained for guiding a reasonable diet in each meal of the user.

In order to ensure the accuracy of calculation, the embodiments of the present application are illustrated with a maximum of three most important chronic diseases selectable by each user for accurate management.

The chronic disease medical record information mainly includes the types of chronic diseases. The chronic diseases targeted by the embodiments of the present application include but are not limited to the following types: diabetes, hypertension, hyperlipidemia, prevention of infection, cardiovascular disease, obesity, anemia, fatty liver, chronic bronchitis, gastritis, cholelithiasis, hyperthyroidism, osteoporosis, etc. Developers can add or adjust the types of chronic diseases based on the requirements for managing other chronic diseases.

The chronic disease history information may include, but is not limited to, the following information of the user: disease type, diagnosis time, severity (mild, moderate, serious, not known), treatment condition (yes or no), used drugs (yes or no), etc. For example, the chronic disease medical record information obtained from a user is that the user has diabetes and hypertension. The chronic disease history information of the same is that the diagnosis of diabetes was three years and eight months ago, with a severity as moderate, the user is receiving treatment, and taking drugs; and the diagnosis of hypertension was ten years ago, with a severity as serious.

(2) Knowing the nutrients: In addition to referring to the public food safety database of the country or region (such as the database of Hong Kong Food Safety Center), the NCMA also has independently developed and established a huge database (Menu Nutrient Database) of various types of Chinese and western cuisines, including the caloric and nutrient information of each food to help the calculation. Eighteen algorithm is used to calculate the amount of eighteen nutrients and calories contained in each food that the user intends to eat in order to derive the recommended serving size of each food.

The Menu Nutrient Database of NCMA covers most types of foods, including the caloric and eighteen nutrient content data of each food. The data includes, but is not limited to, calorie, protein, carbohydrate, fat, dietary fiber, sugar, saturated fat, trans fat, cholesterol, calcium, copper, iron, magnesium, manganese, phosphorus, potassium, sodium, zinc and vitamin C (i.e., Vitamin C).

(3) Pre-meal notice: When a user uses the NCMA on a mobile terminal such as a mobile phone, the user uses the NCMA as a registered member for the nutrition and chronic disease management. After inputting the member number, the meal needs recommendation (including but not limited to: breakfast, lunch, afternoon snack, dinner, night snack, etc.) can be selected, and then, the menu of the meal can be input (including but not limited to handwriting or voice input, or selecting on the apparatus display screen, etc.). The NCMA quickly returns the recommended serving size for each food in the menu. A meal may include the name of one or more foods.

When inputting a menu, the name of the food can be searched by voice or text. If there is no food of the exact same name, the NCMA may forward a similar food to the user for selection.

The NCMA can display the recommended serving size for each food in two manners: (1) a fine method, in the unit of g, ml, etc.; (1) a rough method, in the unit of bowl, cup, spoon, etc. For example, the serving sizes recommended by NCMA for a certain meal of a user according to the menu provided include: shrimps and eggs of half a plate (125 g), tomato and beef of half a plate (130 g), white rice of one bowl (200 g), orange juice of one glass (250 ml), and one apple (71 g).

In order for the user to accurately determine the food serving size, the NCMA may present to the user the size and capacity of the tableware for reference, e.g., a plate, with 20 cm of diameter*2 cm of height, and a capacity of 130 g; a bowl, with 11.5cm of diameter*6 cm of height, and a capacity of 200 g; a cup, with 8 cm of diameter*9.5 cm of height, and a capacity of 250 ml.

If a certain food is recommended in a small amount, it means that the food is not suitable for the user to eat. The user may consider eliminating such food or selecting other food for replacement.

In the embodiments of the present application, the following settings can be made: re-reporting all food is required for each food change. This operation, though cumbersome, can be used to help re-select a proper meal. Certainly, it can also be set according to the use habit as only this type of food is changed specifically each time when the type of food is changed, with the other options remaining unchanged.

(4) Post-meal review: If the user cannot follow the food serving sizes recommended by NCMA, the amount of food taken insufficiently or excessively may simply be entered after the meal for the post-meal review. If the post-meal review list can be designed for the user to enter the actual serving sizes of foods for report, the NCMA can quickly instruct the user how to reduce or increase the same food for compensation in the next meal.

The nutrition and chronic disease management method according to an embodiment of the present application is disclosed in detail below, taking a user suffering from three chronic diseases as an example.

The NCMA can calculate the recommended serving size for each food in the menu based on the following calculation principle:

(1) Knowing the user: acquiring the nutritional requirement information of a user, i.e., the calories and the amounts of eighteen nutrients required by the user each day. However, the nutritional requirement information of each user is not completely the same, and may change by the chronic diseases. In case of a user with a chronic disease, the nutritional requirement information data thereof should be adjusted after the diagnosis as compared with before the diagnosis. It can be defined as the nutritional requirement information (1). By deduction, the body nutritional requirement information after the diagnosis of two chronic diseases is the nutritional requirement information (2), and the body nutritional requirement information data after the diagnosis of three chronic diseases is the nutritional requirement information (3). Therefore, each user should use the calories and eighteen nutrient requirements in the nutritional requirement information (n) as the basis for the nutrient intake requirement according to the n chronic diseases that the user has. The nutritional requirements can be divided into the nutritional requirements per day and the nutritional requirements per meal. In an embodiment of the present application, according to the eating habits of the user, the nutritional requirement information is set as the nutritional requirement information data of the current dining period (i.e., the current meal) of the user.

(2) Knowing the nutrients: The calories and the amounts of eighteen nutrients contained in each food in the menu submitted by the user are taken as the nutrient supply basis of each food, i.e., the calories and the amounts of nutrients contained in the predetermined serving size of each food. The menu may include one or more foods.

(3) Calculation principle: By matching the nutritional requirement information of a certain meal taken by the user with the nutrient supply basis of each food with the eighteen calculations, it is theoretically possible to accurately calculate the appropriate amount of each food, i.e., the recommended serving size of the food, that each user can have during the current meal period.

Accurate calculation would be difficult by using the nutritional software in the prior art due to the great amount of data involved in the calculation. The NCMA used in the embodiments of the present application can perform the accurate calculation based on a menu nutrition database with sufficient data and a predetermined algorithm with the aid of program instructions.

For a user with three chronic diseases, the daily caloric intake required by the user without any chronic disease can be calculated firstly based on the personal physical information data including the personal exercise amount. The Total Daily Energy Expenditure (“TDEE”) of an individual can be calculated using the Harris-Benedict formulae:

Male: [66+1.38*weight (kg)+5*height (cm)−6.8*age]*exercise amount

Female: [65.5+9.6*weight (kg)+1.9*height (cm)−4.7*age]*exercise amount.

Then, the nutritional data is then used to calculate the amounts of eighteen nutrients required by the user each day. Lastly, according to the eating habits of the user, the calories and nutrients required by the user during the current dining period can be calculated to provide the personal nutritional requirement information of the user with no chronic diseases.

Since the user suffers from the chronic diseases, it is necessary to calculate the limits of nutritional requirement information of the user for each chronic disease based on medical nutrition. Therefore, after obtaining the user's personal nutritional requirement information in the absence of a chronic disease, the personal nutritional requirement information needs to be adjusted to the nutritional requirement information (1) in order to be adapted to the dietary requirements for the first chronic disease of the user. If there is a second chronic disease, the nutritional requirement information (1) needs to be adjusted to the nutritional requirement information (2) in order to be adapted to the second chronic disease. If there is a third chronic disease, the nutritional requirement information (2) needs to be adjusted to the nutritional requirement information (3) in order to be adapted to the third chronic disease. The nutritional requirement information (3) is the final nutritional requirement information of this user with three chronic diseases.

The three nutrient types that most affect the user's chronic diseases are determined from the user's final nutritional requirement information. The amounts of these three nutrients required by the user, and the calories required can be taken as the four top priorities determining the recommended serving size of the user during the current dining period as a pre-meal forecast calculation.

For example, if one of the chronic diseases of the user is diabetes, the limits posed by the diabetes on the user's nutritional requirement information are as follows:

Total calories: limiting the total calories, only taking the minimum necessary calories until the body weight is slightly below ideal weight;

Carbohydrates: daily carbohydrate of 250-350 g, 150-200 g for obese users, but no less than 100 g to prevent the ketoacidosis;

Proteins: high protein consumption, adults supplied at 1.0-1.5 g/kg per day, children, pregnant women, lactating women and users of poor nutrition calculated according to the standard of 1.5-2.0 g/kg;

Fat: limiting the fat intake, 0.6-1.0 g/KG per day;

Cholesterol: dietary cholesterol intake of lower than 300 mg/d;

Dietary fiber and trace elements: dietary fiber of 40 g/d, supplementing calcium of no less than 1000 mg/d, and zinc of 10-15 mg/d.

After the user's final nutritional requirement information is calculated, the recommended serving size is calculated using the calories and eighteen nutrients contained in each food according to the menu provided by the user.

For example, the lunch menu provided by the user suffering from three chronic diseases includes apple (with skin), Chinese white rice (steamed), shrimps and eggs, Chinese sour and spicy soup, and parsley and chicken. The predetermined serving sizes of various foods are also provided. According to the information obtained from the menu nutrition database or the public food safety database of the country or region, the caloric and nutrient information of various foods in the predetermined serving sizes can be listed as follows:

| | | | | | | | | | | | | |
|---|---|---|---|---|---|---|---|---|---|---|---|---|
| | Predetermined | | | | | Dietary | | Saturated | Trans | | | |
| | Serving Size | Energy | Protein | Carbohydrate | Fat | Fiber | Sugar | Fat | Fat | Cholesterol | Calcium | Copper |
| Food Name | (g/ml) | (Kcal) | (g) | (g) | (g) | (g) | (g) | (g) | (g) | (mg) | (g) | (g) |

Lunch

| | | | | | | | | | | | | |
|---|---|---|---|---|---|---|---|---|---|---|---|---|
| Apple | 60 | 31.20 | 0.16 | 8.29 | 0.10 | 1.44 | 6.23 | 0.02 | NA | 0 | 3.60 | 0.02 |
| (with skin) | | | | | | | | | | | | |
| Chinese | 150 | 226.50 | 4.80 | 50.82 | 0.41 | 1.35 | NA | NA | NA | NA | 7.50 | 0.07 |
| white rice | | | | | | | | | | | | |
| (steamed) | | | | | | | | | | | | |
| Shrimps | 150 | 345.00 | 18.50 | 3.90 | 27.00 | 0 | 0.41 | 4.65 | 0.18 | 525.00 | 63.00 | 0.10 |
| and eggs | | | | | | | | | | | | |
| Chinese | 100 | 39.00 | 2.58 | 4.35 | 1.21 | 0.50 | 0.42 | 0.23 | 0 | 21.00 | 18.00 | 0.03 |
| sour and | | | | | | | | | | | | |
| spicy soup | | | | | | | | | | | | |
| Parsley and | 150 | 136.50 | 11.10 | 3.75 | 8.70 | trace | 1.80 | 1.26 | 0.06 | 55.50 | 31.50 | trace |
| chicken | | | | | | | | | | | | |
| Total amount | 610 | 778.20 | 38.14 | 71.11 | 37.42 | 3.29 | 8.86 | 6.16 | 0.24 | 601.50 | 124.6 | 0.22 |
| in lunch | | | | | | | | | | | | |
| Nutritional | | 530 Kcal | 30 g | | | 8 g | | | | 100 mg | | |
| requirement | | | | | | | | | | | | |
| information | | | | | | | | | | | | |

Then, the three types of nutrients that most affect the various chronic diseases of the user determined according to the chronic disease types of the user are the protein, dietary fiber and cholesterol, the amounts of which required in the nutritional requirement information are respectively 30 g, 8 g and 100 mg. These three nutrients together with the calories of 530 Kcal in the nutritional requirement information serve as the four top priorities for continuing to calculate the user's recommended serving size.

A predetermined upper and lower threshold values of adjustment for the predetermined serving size of each food need to be obtained before the predetermined serving size is adjusted based on the four top priorities. The purpose of setting the predetermined upper and lower threshold values of adjustment is to avoid adding or reducing certain ingredient to an unreasonable amount in the process of counter-claim calculation based on the top priorities.

The NCMA then matches the nutritional requirement information with the predetermined serving sizes four times according to the required serving sizes of protein, cholesterol, dietary fiber and calories. It is to be noted that in the four adjustments, the adjustment of the predetermined serving size of each food according to the calories required by the user is carried out at the end of the four top priorities, so as to make the total calories contained in the recommended serving sizes equal to the calories required by the user. The recommended serving size of each food is obtained after adjustment. The recommended serving size is the amount that best satisfies the nutritional requirement information of the user, i.e., the result of a pre-meal notice.

In addition, the NCMA can find the average daily nutrient intake from the monthly total dietary record of the user, and compare it with the nutritional requirement information of the user, so as to analyze whether the caloric intake of the user and the amounts of eighteen nutrients are sufficient or excessive, and hint the user whether an improvement is necessary.

The NCMA may also provide the education and help functions. Each member is educated on the chronic disease information, such as causes, detection, prevention, dietary care, nursing and monitoring, by the short texts or films regularly pushed to the user. It is also possible to push to the user other interesting and useful information related to the healthy life, e.g., how to exercise, how to maintain the desired weight, etc. The NCMA may also periodically remind the members on relevant medical examinations to check the physical condition.

If a user is found to have long-term insufficient intake of certain nutrients, such as protein, calcium, iron, vitamins A, D, B, etc. the NCMA may teach the user how to supplement them with food or supplements.

In the technical solution of the nutrition and chronic disease management method of the embodiments of the present application, the NCMA is used for providing the dietary guidance to the users with chronic diseases. It is simple and accurate, reacts instantly and comprehensively to dietary recommendation requests, and help the users to continuously establish and improve the healthy lifestyles. It can promote the health more accurately, and prevent and control the chronic diseases as compared with the prior art.

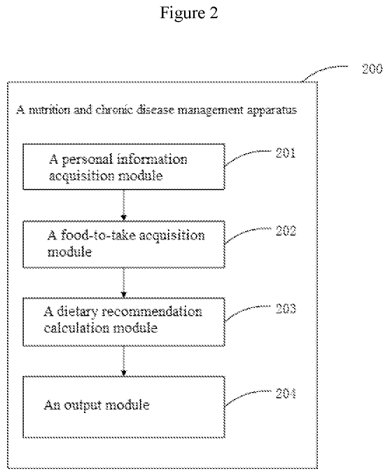

FIG. 2 is a schematic view of the structure of a nutrition and chronic disease management apparatus disclosed in an embodiment of the present application. As shown in FIG. 2, a nutrition and chronic disease management apparatus 200 according to an embodiment of the present application comprises: a personal information acquisition module 201, a food-to-take acquisition module 202, a dietary recommendation calculation module 203 and an output module 204.

Amongst them, the personal information acquisition module 201 can be used for acquiring the personal profile information of the user, and calculating the nutritional requirement information of the user according to the personal profile information, wherein the personal profile information includes the chronic disease information of the user, and the nutritional requirement information includes the calories required by the user and the amounts of nutrients affecting the chronic diseases of the user;

The food-to-take acquisition module 202 can be used for acquiring the name of one or more foods to be taken by the user;

The dietary recommendation calculation module 202 can be used for obtaining caloric and nutrient content information of each of the one or more foods according to the name of the one or more foods, and calculating a recommended serving size of each food according to the nutritional requirement information and the caloric and nutrient content information of each food; and

The output module 204 can be used for outputting the recommended serving size.

Amongst them, the personal profile information further includes personal physical information, the personal physical information including, but not limited to, age, gender, height, weight, personal exercise amount, and eating habit information of the user; and the calories required by the user are calculated based on the personal physical information.

The nutritional requirement information is for a current meal period determined according to the user's daily nutritional requirement information and the user's eating habits.

The chronic disease information includes the chronic disease medical record information and the chronic disease history information, wherein the chronic disease medical record information includes, but is not limited to, the medical record data of one to three chronic diseases of the user. The chronic disease history information includes, but is not limited to, the disease type, diagnosis time, severity, treatment condition, and used drugs of the chronic disease of the user.

In the nutrition and chronic disease management apparatus according to an embodiment of the present application, the food-to-take acquisition module 202 is also applied to obtaining a predetermined serving size of each of the one or more foods when obtaining the name of the one or more foods to be taken by the user; the dietary recommendation calculation module 202 is also applied to adjusting the predetermined serving size according to the nutritional requirement information and the caloric and nutrient content information of each food, so as to obtain the recommended serving size.

In the embodiments of the present application, the nutrients affecting the chronic diseases of the user include, but are not limited to, at least eighteen of the following: protein, carbohydrate, fat, dietary fiber, sugar, saturated fat, trans fat, cholesterol, calcium, copper, iron, magnesium, manganese, phosphorus, potassium, sodium, zinc and vitamin C.

In the nutrition and chronic disease management apparatus according to an embodiment of the present application, the personal information acquisition module 201 is also applied to: selecting three nutrients most affecting the chronic diseases of the user from the nutrients affecting the chronic diseases of the user, and using the required amounts of the three nutrients and the calories required by the user as four top priorities for guiding the user's meal.

In the nutrition and chronic disease management apparatus according to an embodiment of the present application, the food-to-take acquisition module 202 is also applied to: obtaining a predetermined serving size of each of the one or more foods when obtaining the name of the one or more foods to be taken by the user, and the dietary recommendation calculation module 202 is also applied to: adjusting the predetermined serving size of each food respectively according to the four top priorities to obtain the recommended serving size of each food.

Among them, the dietary recommendation calculation module 202 is also applied to adjusting the predetermined serving size of each food according to the calories required by the user at the end of the four top priorities, so as to make the total calories contained in the recommended serving sizes equal to the calories required by the user.

In an embodiment of the present application, the food-to-take acquisition module 202 is also applied to obtaining the predetermined upper and lower threshold values of adjustment for the predetermined serving size of each food, when the predetermined serving size of each food is obtained. The purpose of setting the predetermined upper and lower threshold values of adjustment is to avoid adding or reducing certain ingredient to an unreasonable amount in the process of counter-claim calculation based on the top priorities.

In an embodiment of the present application, the dietary recommendation calculation module 202 is also applied to obtaining the caloric and nutrient content information of each food from a predetermined recipe nutrient database according to the name of the one or more foods.

In an embodiment of the present application, the nutrition and chronic disease management apparatus further comprises a supervision and recommendation module (not shown) for recording the serving sizes taken by the user greater or smaller than the recommended serving sizes after the meal, so as to guide the user's subsequent meals.

The supervision and recommendation module is also applied to generating a dietary record report of the user according to a predetermined first period (e.g., one month as a period), and determining whether the user has an insufficient or excessive nutrient intake during the predetermined first period, so as to determine whether to give a dietary remedy recommendation. In addition, the supervision and recommendation module is also applied to providing a dietary recommendation to the user according to a predetermined second period (e.g., a quarter or year as a period).

The nutrition and chronic disease management apparatus of an embodiment of the present application may be the aforementioned nutrition and chronic disease management application NCMA, which may be installed on various types of terminal equipment including the mobile phone.

According to the technical solution of the nutrition and chronic disease management apparatus in an embodiment of the present application, the dietary guidance in the embodiment of the present application can be specially designed for the users with chronic diseases, and can establish and improve the healthy life mode of the users with chronic diseases. The large database resources can be guaranteed by the database of independently developed recipes, including the combination of Chinese and western cuisines, and the public food safety database of the country or region (such as the database of Hong Kong Food Safety Center in Hong Kong). In addition, when the technical solutions of the embodiments of the present application provide a dietary recommendation, the caloric requirements and various nutrient requirements of a user are considered comprehensively, e.g., at least eighteen types of nutrients may be included. Also, more accurate and scientific dietary guidance can be provided by focusing on the three nutrients that have the greatest impact on the user's chronic diseases. The dietary recommendation provided in the technical solution of the present application is an accurate calculation result rather than a simple estimation. The recommendation can be quickly finished according to the menu provided by the user, and the user does not need to wait for a long time. The technical solution of the present application can not only provide the pre-meal recommendation, but also perform the post-meal supervision. It can supervise the diet of a user for long term, and pertinently provide the personalized education for different users.

Embodiments of the present application also provide a storage medium storing a program. When the program is executed, the instructions for implementing the nutrition and chronic disease management method as in the present application can be executed.

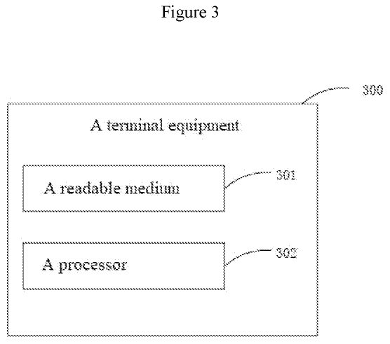

FIG. 3 is a schematic view of the structure of a terminal equipment disclosed in an embodiment of the present application. As shown in FIG. 3 of the present application, the embodiment of the present application also provides a terminal equipment 300 comprising a readable medium 301 storing program instructions and a processor 302. The processor 302 implements the nutrition and chronic disease management method as disclosed in the embodiment of the present application when running the program instructions. Amongst them, the terminal equipment includes, but is not limited to, various terminal equipment such as a mobile phone, a laptop, a tablet computer, a desktop computer, and a vehicle-mounted computer which can be installed with an NCMA.

In the present application, the embodiments are provided in a progressive manner, with each embodiment focusing on an aspect different from other embodiments. The identical or similar parts among the embodiments can be referred to in each other. For the apparatus disclosed in the embodiments, since it corresponds to the method disclosed in the embodiments, its elaboration is relatively simple. Please refer to the elaboration on the method for the relevant parts.

It is further noted that the use of relational terms such as “first” and “second” and the like in the application documents of the present application is only intended to distinguish one entity or operation from another entity or operation, without necessarily requiring or implying any actual such relationship or sequence among such entities or operations. Furthermore, the terms “comprising”, “including”, or any other variation thereof, are intended to cover a non-exclusive inclusion, such that a process, method, article, or apparatus that comprises a list of elements does not include only those elements, but may include other elements not expressly listed, or inherent to such process, method, article, or apparatus. In the absence of more limitations, an element limited by the expression of “comprising a . . . ” does not preclude the existence of additional identical elements in the process, method, article, or apparatus that comprises this element.

The technical solutions provided in the present application are elaborated in detail above. The principles and embodiments of the present application have been elaborated herein using specific examples, the above examples are only intended to aid in understanding the methodology and core concepts of the present application. It should be noted that for those skilled in the art, improvements and amendments can be made to the present application if not deviating from the principles of the present application. Such improvements and amendments also fall within the protection scope of the present application.

1. A nutrition and chronic disease management method, comprising:
acquiring personal profile information of a user, and calculating nutritional requirement information of the user according to the personal profile information, wherein the personal profile information includes chronic disease information of the user, and the nutritional requirement information includes the calories required by the user and the amounts of nutrients affecting the chronic diseases of the user;
obtaining a name of one or more foods to be taken by the user;
obtaining caloric and nutrient content information of each of the one or more foods according to the name of the one or more foods, and calculating a recommended serving size of each food according to the nutritional requirement information and the caloric and nutrient content information of each food; and
outputting the recommended serving size.

2. The nutrition and chronic disease management method according to claim 1, wherein the personal profile information further includes personal physical information, the personal physical information including age, gender, height, weight, personal exercise amount, and eating habit information of the user; and the calories required by the user are calculated based on the personal physical information.

3. The nutrition and chronic disease management method according to claim 1, wherein the nutritional requirement information is for a current meal period determined according to the user's daily nutritional requirement information and the user's eating habits.

4. The nutrition and chronic disease management method according to claim 1, wherein the chronic disease information includes chronic disease medical record information and chronic disease history information, wherein the chronic disease medical record information includes the medical record data of one to three chronic diseases of the user, and the chronic disease history information includes the disease type, diagnosis time, severity, treatment condition, and used drugs of the chronic disease of the user.

5. The nutrition and chronic disease management method according to claim 1, wherein the method further comprises:
obtaining a predetermined serving size of each of the one or more foods when obtaining the name of the one or more foods to be taken by the user, and calculating a recommended serving size of each food according to the nutritional requirement information and the caloric and nutrient content information of each food, comprising:
adjusting the predetermined serving size according to the nutritional requirement information and the caloric and nutrient content information of each food to obtain the recommended serving size.

6. The nutrition and chronic disease management method according to claim 1, wherein the nutrients affecting the chronic diseases of the user include at least eighteen of the following: protein, carbohydrate, fat, dietary fiber, sugar, saturated fat, trans fat, cholesterol, calcium, copper, iron, magnesium, manganese, phosphorus, potassium, sodium, zinc and vitamin C.

7. The nutrition and chronic disease management method according to claim 1, wherein the method further comprises: selecting three nutrients most affecting the chronic diseases of the user from the nutrients affecting the chronic diseases of the user, and using the required amounts of the three nutrients and the calories required by the user as four top priorities for guiding the user's meal.

8. The nutrition and chronic disease management method according to claim 7, wherein the method further comprises:
obtaining a predetermined serving size of each of the one or more foods when obtaining the name of the one or more foods to be taken by the user, and calculating a recommended serving size of each food according to the nutritional requirement information and the caloric and nutrient content information of each food, comprising:
adjusting the predetermined serving size of each food respectively according to the four top priorities to obtain the recommended serving size of each food.

9. The nutrition and chronic disease management method according to claim 8, wherein the adjustment of the predetermined serving size of each food according to the calories required by the user is carried out at the end of the four top priorities, so as to make the total calories contained in the recommended serving sizes equal to the calories required by the user.

10. The nutrition and chronic disease management method according to claim 5, wherein when the predetermined serving size of each food is obtained, the predetermined upper and lower threshold values of adjustment for the predetermined serving size of each food are also obtained.

11. The nutrition and chronic disease management method according to claim 1, wherein the caloric and nutrient content information of each of the one or more foods obtained according to the name of the one or more foods includes: the caloric and nutrient content information of each food obtained from a predetermined recipe nutrient database according to the name of the one or more foods.

12. The nutrition and chronic disease management method according to claim 1, further comprising: after the meal, recording the serving sizes taken by the user greater or smaller than the recommended serving sizes, so as to guide the user's subsequent meals.

13. The nutrition and chronic disease management method according to claim 1, further comprising: generating a dietary record report of the user according to a predetermined first period, and determining whether the user has an insufficient or excessive nutrient intake during the predetermined first period, so as to determine whether to give a dietary remedy recommendation.

14. The nutrition and chronic disease management method according to claim 1, further comprising: providing a dietary recommendation to the user according to a predetermined second period.

15. A nutrition and chronic disease management apparatus, comprising:
a personal information acquisition module for acquiring the personal profile information of the user, and calculating the nutritional requirement information of the user according to the personal profile information, wherein the personal profile information includes the chronic disease information of the user, and the nutritional requirement information includes the calories required by the user and the amounts of nutrients affecting the chronic diseases of the user;
a food-to-take acquisition module for acquiring the name of one or more foods to be taken by the user;
a dietary recommendation calculation module for obtaining caloric and nutrient content information of each of the one or more foods according to the name of the one or more foods, and calculating a recommended serving size of each food according to the nutritional requirement information and the caloric and nutrient content information of each food; and
an output module for outputting the recommended serving size.

16. The nutrition and chronic disease management apparatus according to claim 15, wherein the personal profile information further includes the personal physical information, the personal physical information including age, gender, height, weight, personal exercise amount, and eating habit information of the user; and the calories required by the user which are calculated based on the personal physical information.

17. The nutrition and chronic disease management apparatus according to claim 15, wherein the nutritional requirement information is for the current meal period determined according to the user's daily nutritional requirement information and the user's eating habits.

18. The nutrition and chronic disease management apparatus according to claim 15, wherein the chronic disease information includes chronic disease medical record information and chronic disease history information, wherein the chronic disease medical record information includes the medical record data of one to three chronic diseases of the user, and the chronic disease history information includes the disease type, diagnosis time, severity, treatment condition, and used drugs of the chronic disease of the user.

19. The nutrition and chronic disease management apparatus according to claim 15, wherein the food-to-take acquisition module is also applied to:
obtaining a predetermined serving size of each of the one or more foods when obtaining the name of the one or more foods to be taken by the user, and the dietary recommendation calculation module is also applied to:
adjusting the predetermined serving size according to the nutritional requirement information and the caloric and nutrient content information of each food, so as to obtain the recommended serving size.

20. The nutrition and chronic disease management apparatus according to claim 15, wherein the nutrients affecting the chronic diseases of the user include at least eighteen of the following: protein, carbohydrate, fat, dietary fiber, sugar, saturated fat, trans fat, cholesterol, calcium, copper, iron, magnesium, manganese, phosphorus, potassium, sodium, zinc and vitamin C.

21. The nutrition and chronic disease management apparatus according to claim 15, wherein the personal information acquisition module is also applied to: selecting three nutrients most affecting the chronic diseases of the user from the nutrients affecting the chronic diseases of the user, and using the required amounts of the three nutrients and the calories required by the user as four top priorities for guiding the user's meal.

22. The nutrition and chronic disease management apparatus according to claim 21, wherein the food-to-take acquisition module is also applied to:
obtaining a predetermined serving size of each of the one or more foods when obtaining the name of the one or more foods to be taken by the user, and the dietary recommendation calculation module is also applied to:
adjusting the predetermined serving size of each food respectively according to the four top priorities to obtain the recommended serving size of each food.

23. The nutrition and chronic disease management apparatus according to claim 22, wherein the dietary recommendation calculation module is also applied to adjusting the predetermined serving size of each food according to the calories required by the user at the end of the four top priorities, so as to make the total calories contained in the recommended serving sizes equal to the calories required by the user.

24. The nutrition and chronic disease management apparatus according to claim 19, wherein the food-to-take acquisition module is also applied to obtaining the predetermined upper and lower threshold values of adjustment for the predetermined serving size of each food, when the predetermined serving size of each food is obtained.

25. The nutrition and chronic disease management apparatus according to claim 15, wherein the dietary recommendation calculation module is also applied to obtaining the caloric and nutrient content information of each food from a predetermined recipe nutrient database according to the name of the one or more foods.

26. The nutrition and chronic disease management apparatus according to claim 15, further comprising a supervision and recommendation module for recording the serving sizes taken by the user greater or smaller than the recommended serving sizes after the meal, so as to guide the user's subsequent meals.

27. The nutrition and chronic disease management apparatus according to claim 26, wherein the supervision and recommendation module is also applied to generating a dietary record report of the user according to a predetermined first period, and determining whether the user has an insufficient or excessive nutrient intake during the predetermined first period, so as to determine whether to give a dietary remedy recommendation.

28. The nutrition and chronic disease management apparatus according to claim 26, wherein the supervision and recommendation module is also applied to providing a dietary recommendation to the user according to a predetermined second period.

29. A storage medium storing a program, characterized in that the program comprises instructions for implementing the method according to claim 1.

30. A terminal equipment comprising a readable medium storing program instructions and a processor, characterized in that the processor implements the method according to claim 1 when running the program instructions.
